(19) United States
(12) Patent Application Publication
(10) Pub. No.: US 2013/0280792 A1
(43) Pub. Date: Oct. 24, 2013

(54) PROCESSING EQUIPMENT FOR ORGANIC WASTE

(71) Applicant: Gennadiy Chernov, Horousanky (CZ)

(72) Inventor: Gennadiy Chernov, Horousanky (CZ)

(73) Assignee: KEY GROUP HOLDING S.R.O., Prague 4 (CZ)

(21) Appl. No.: 13/991,465

(22) PCT Filed: Feb. 22, 2011
(86) PCT No.: PCT/IB11/50733

§ 371 Date: Jun. 19, 2013

(30) Foreign Application Priority Data
Nov. 8, 2010 (CZ) PV 2010-807

(51) Int. Cl. C12N 1/12

(52) U.S. Cl. CPC C12N 1/12
USPC 435/257.1; 435/262; 435/289.1; 252/180

(57) ABSTRACT

A facility for complex processing of organic waste. The facility includes a waste preparation module, which includes waste separation, waste mixing and biotunnel. The facility further includes a module for waste pyrolysis and gasification, including a reactor for pyrolysis and partial gasification connected to a gasification reactor. The reactor for pyrolysis and partial gasification and the reactor for gasification contain melts of salts. Further, the reactors are connected with a gas storage tank which is connected with a reactor for power and heat generation.

### TECHNICAL FIELD

The invention deals with processing of organic waste and with equipment for its processing and with utilization of processed products, during which the waste is processed completely to basic elements, which in turn can be utilized as a raw material for further production, while certain products will be used to feed livestock, or a biological suspension will be used for biological recovery of water reservoirs and for eradication of blue-green algae, or a biological suspension will be used for water treatment.

### CURRENT TECHNICAL CONDITION

So far the organic waste disposal has been conducted using equipment for e.g. plasma incineration or thermal cracking; eventually the waste is disposed of in fermentation gas station or compost plant. The biggest disadvantage of the above mentioned installations is the fact, they do not allow disposal of all produced wastes or the result is not waste utilization for energy generation or as raw material for further utilization. This creates another problem of treatment of raw materials output by the above mentioned devices. The disposed waste thus leaves smaller part of the raw materials, which become further waste, not being disposed off and burdening the environment. During combustion of these components emission pollution of the environment occurs. The products of known methods of organic waste disposal can be utilized neither to feed livestock, for biological recovery of water reservoirs, nor is the created suspension utilized for water treatment. The composition of products from organic waste disposal made those methods of utilization impossible, because they were produced by a completely different technology and their final composition was not suitable for utilization for the above mentioned purposes.

### SUBJECT MATTER OF INVENTION

The indicated deficiencies are corrected by the method of organic waste processing in accordance with this invention, subject matter of which lies in the fact, that organic waste is transferred into the accessory module. Separation of glass and metal will take place there, as well as mincing and stabilization of food remains and other waste. Stabilization takes place through supply of air aerobic bacterium. The waste is fed through a biotunnel, where the temperature is raised and water evaporates, which is further devaporated and used for other purposes. After the waste passes the biotunnel, it is compressed into pellets, which are transported by carbon dioxide into the loading device, from which they are transferred by an auger into the dispenser, where the waste is compressed again and after that it is discharged by carbon dioxide into the reactor. This reactor contains melt of various salts with temperature from 900 to 1000° C. Pyrolysis occurs in the reactor, producing particular elements C, H, N, S, and O2 and creates methane and other gases. The composition of gases is given by the pressure in the thermal cracker. The methane volume is modified by external pressure regulation. Metal oxides, which are contained in the pyrolysis waste, are reduced to pure metals using a melt. The top part of the reactor contains gases and the bottom part carbon and pure metals. The level of melt is also regulated through pressure, while being mechanically stirred. After conclusion of thermal cracking, while the cracking time is determined by a calculation, the valve connecting the pipes opens and the melt is transferred under pressure into the reactor. The reactor is connected by two tubes with gasification reactor. The gases from the top part of the reactor are transferred into the bottom part of the reactor through pipes. The melt from the reactor goes through pipes into the bottom part of the reactor. Steam from the steam generator is being fed into the top pipe. Gasification occurs in the reactor in the presence of steam, under temperatures from 940 to 1000° C., while H, CO, CO2, H2S, NH4, CH4 and other hydrocarbons are created, according to the presence of elements. The volume of methane and other hydrocarbons, up to butane, is controlled by the means of pressure. The gasses pass into the top part of the reactor chamber and stream through pipes into the cyclone separator, where it is mechanically purified. Mechanic particles return into the feeder. Pure gas streams through pipes into the feeder, where it is analyzed for presence of dangerous gases FH or dioxins. P\check{r}i nalezení nebezpe\check{c}ných plyn [symbol] se ventil na potrubí zav\check{r}e a otev\check{r}e se ventil na potrubí. The contaminated gas returns to the reactor, supply of waste from feeder is cut and hydrate oxides are supplied from the feeder for gas quality correction into the feeder for neutralization. The process then repeats. When gasification is concluded, manifold valve opens and part of the increased level of melt will automatically pass into the filtration device containing zirconium filter with apertures of different diameters, through which the melt passes and where the metals are captured. Saturated filter is placed into an induction furnace, where the filter is pulverized in an inert environment and the powder is used as a raw material for further processing. The melt passes from the device through manifold into the pump chamber, from which it is pushed though manifold into the reactor. This completes the cycle. Sewage, also considered a source of organic waste, is transferred into a reservoir, where it is oxidized using anaerobic bacterium, producing sludge and technical water. The sludge can be mixed into the liquid waste reservoir either alone or together with solid waste.

The above mentioned deficiencies are remedied by the device for complete processing of organic waste, comprising of the waste preparation module, which includes waste separation, waste mixing and biotunnel, pyrolysis module and waste gasification, comprising of pyrolysis and partial gasification reactor, connected with the gasification reactor, where the liquid waste reservoir is mounted above the pyrolysis and partial gasification reactor, serving as dispenser for complex processing of organic waste, in correspondence with this invention, whose subject matter lies in the fact, that pyrolysis and partial gasification reactor and gasification reactor contain melt of salts and are connected to the melt filtration device with heater. On one side the device is connected to a module for metal smelting on one side and pump chamber for transfer of the salt melt into the pyrolysis and partial gasification reactor on the other. The gasification reactor is connected by a manifold to a cyclone for mechanical separation and connecting pipe connects the pump chamber with the pyrolysis and partial gasification reactor. The manifold also connects the above mentioned reactor with the gasification reactor. The cyclone is connected with the damping reservoir for storage of contaminated synthesis gas, which is connected with the pyrolysis and partial gasification reactor by a manifold for transfer of raw gas and with the synthesis gas preparation module for different purposes. Manifold from the synthesis gas preparation module leads into the module for CO2 extraction from exhaust gas from energy and fuel production. One manifold also leads from the synthesis gas preparation module into the power and heat generation module and another into the elemental sulfur production, while the CO2 extraction module is connected by a branching manifold into the bio-technological product production module, comprising of the device for fodder suspension production, based on the Parachlorella KIEG 1904, device for algae production for the purpose of watersheds recovery and eradication of blue-green algae, and device for dry biomass production. The dry biomass production device is connected with the device for dry algae oil production, which is further connected with the device for production of fatty acids' esters from algae oil, while the device for production of fatty acids' esters from algae oil is connected with the block for glycerol extraction, from which manifold leads into the power and heat generation module.

Bio-technological products, containing Parachlorella KIEG 1904 algae, will be used for feeding livestock.
Biological suspensions will be used for bio-recovery of water reservoirs and for blue-green algae disposal.
Biological suspensions will be used for water treatment.
The device will serve for production of biological suspension based on the Parachlorella KIEG 1904 strain.
The device will be used for complex utilization of technologies for waste processing and bio-technologies through building of subterranean compounds in residential areas (minimum 5000 residents, maximum 20 000 residents), i.e. Local Energetic Compounds (LEC).

### OVERVIEW OF IMAGES IN DRAWING

The invention will be described in detail in drawings, where the image 1 shows connection diagram of particular parts of the device. Image 2 shows connection for synthesis gas preparation for different purposes, image 3 shows the parts of the device for production of the fodder suspension based on Parachlorella KIEG 1904, image 4 shows parts of the device for production of the suspension for watersheds recovery and eradication of blue-green algae, and image 5 shows the device for dry biomass production.

### EXAMPLES OF INVENTION IMPLEMENTATION

1. The invention, according to image 1, comprises of the module 1 for waste preparation, which includes waste separation, waste mixing and biotunnel. Module 2 for pyrolysis and waste gasification comprises of the reactor 2.1 for pyrolysis and partial gasification, connected with the gasification reactor 2.2. The liquid waste reservoir 2.3, serving as a dispenser, is located above the reactor 2.1. Reactor 2.1 and reactor 2.2 are interconnected with the device 2.4 for melt filtration with the heater 2.7. On one side the device 2.4 is connected to a module 10 for metal smelting on one side and pump chamber 2.9 for transfer of the salt melt into reactor 2.1 on the other. The reactor 2.2 is fed through the connecting manifold 2.6 for the cyclone 2.5 for mechanical separation. The connecting manifold 2.6 also connects the pump chamber 2.9 with the reactor 2.1 and also reactor 2.1 with reactor 2.2. The cyclone 2.5 is connected with the damping reservoir 2.8 for storage of untreated synthesis gas, which is connected with reactor 2.1 for transfer of raw gas and with module 3 for the synthesis gas preparation for different purposes. Manifold connects module 3 with the module 4. Manifold also leads from module 3 into the module 5 for power and heat generation and a manifold connects it with module 6 for elemental sulfur production. Module 4 is connected by a branching manifold with the module for production of bio-technological products comprising of device 7.1 for production of fodder suspension based on the Parachlorella KIEG 1904, device 7.2 for production of suspension for water reservoirs recovery and blue-green algae disposal, and of device 7.3 for dry biomass production. The device 7.3 is connected with the device 8 for production of oil from dry algae biomass, which is further connected with the device 9 for production of esters from fatty acids from algae oil. Device 9 is connected with the block 9.1 for glycerol extraction, from which a manifold leads into module 5 for power and heat generation.
2. Livestock fodder description

Experiments with utilization of the Parachlorella KIEG 1904 were conducted on several poultry ranches in EU. The experiments were launched in breeding reproduction installation on a poultry ranch. Hens from the control group (n=2 thousand) were not receiving the preparation. Hens from the experimental group (n=2 thousand) were receiving the chlorella suspension—dosage 40-50 ml during 30 days. Feeders for breeding poultry were installed near the passing trough and each day 80-100 l of suspension have been added. According to calculations one hen consumed 40-50 ml.

The main task was to gain from the hens high-quality eggs for incubation. All hens were in a good condition during the experiment. Compliance with zoo technical standards for breeding, balanced fodder and permanent veterinary supervision allowed effective poultry production. Due to chlorella feeding the reproduction potential of the hens improved.

2783 eggs from the experimental group and 3625 eggs from the control group were used for the purposes of incubation, from which hatched 2310 and 2411 chickens respectively, representing 83.0 and 66.5% respectively. Chickens hatched from eggs from the experimental group were fed 5 ml of preparation for 7 days, than 30 ml for 35 days. The use of chlorella did not affect the current technology of chicken breeding and breeding poultry. Weekly weighing of chicken has been performed. Each coop placed 14 chickens. The experiment included total of 1848 chickens in the control and experimental groups.

The chickens hatched from the eggs from the experimental group grew rapidly and after the first week of life they surpassed their contemporaries from the control group (Table 1).

During the entire period of chickens' feeding the survival rate reached 98.05% in the experimental group, 92.97% in control group, smaller quantity required slaughter for sanitary reasons −2.4% (experimental) and 9.7% (control).

Therefore the mortality of poultry fed by chlorella was 3.6-4 times lower.

During the experiment (42 days) the difference between the live weights compared to the control group amounted at 10.7%, slaughter weight 19.9%. Out of the processed poultry meet from control group eighty percents of the overall volume of liver have been discarded; in the experimental group that index totaled 3%

TABLE 1
Chicken live weight and survival rate

| | | Group | |
|---|---|---|---|
| Value | | Control | Experimental |
| Live weight (g) after | 7 days | 114.60 | 127.10 |
| | 42 days | 1578.00 | 1747.00 |
| Survival rate (%) after | 7 days | 99.89 | 99.95 |
| | 42 days | 92.97 | 98.05 |
| Slaughter for sanitary reasons (%) after | 7 days | 0.16 | 0.06 |
| | 42 days | 9.70 | 2.40 |

For the purposes of further experiment they used a poultry ranch with average daily live-weight gain exceeding 50 g, with survival rate 96-97% and with low fodder conversion value. The experiment has been conducted under production conditions. Poultry from one facility participated on the experiment—23 760 pcs—which has been daily fed chlorella suspension from 5 ml starting day 5 after hatching up to 30 ml for 37 days until slaughter. During the poultry feeding total of 12 125 l of chlorella suspension has been used. The remaining facilities with total of 115 860 pcs of poultry were control facilities. Chlorella suspension affected meat quality indicators of the slaughtered poultry. Bio-chemical analysis of blood, muscle matter and bones of the poultry's experimental group showed improvement of all values by 15-25%, as compared with control groups. It also has to be mentioned that during the experiment poultry did not use any antibiotics and vitamins. As a result of feeding chlorella algae infectious deceases of poultry were substantially less frequent.

The algae have similar effects on pigs, livestock, fish and bees.

3. Eradication of blue-green algae

Due to increasing anthropogenic load on the environment many water reservoirs and rivers underwent changes associated with deteriorating water quality, degradation of biocenosis and structural transformations of ichthyofauna. With respect to the utilization of water reservoirs as sources of potable water in surrounding communities the deterioration of water quality due to functional changes in water reservoir ecosystem is particularly alarming.

Dominating propagation of blue-green algae, causing long-term algae bloom with accumulation of superfluous biomass, causes technical difficulties in water supply for municipal water distribution networks and also causes deterioration of chemical composition of water and hygienic markers.

Algae bloom in reservoirs is caused by three strains of blue-green algae: Aphanizomenon, Anabaena and Microcystis.

Biological approach has been introduced to solve the problem of algae bloom implying destructuralization of phytoplankton, during which the ratio of blue-green algae to green algae should shift towards the green algae. These are known antagonistic relations developed between green-blue and green algae in plankton. The domination of green algae in a watershed prevents mass propagation of green-blue algae and prevents the algae bloom. To improve the role of green algae in struggle against green-blue algae the treatment of a water reservoir with Parachlorella MEG 1904 is being introduced. Due to plankton-like features this strain shows apparent antagonism to green-blue algae Aphanizomenon, Anabaena and Microcystis.

The treatment of water reservoirs with plankton strains of chlorella creates conditions for normalization of hydro-biological conditions in the watersheds and prevents propagation of algae bloom.

The experiments to establish the effect of green algae on propagation of green-blue algae in water reservoirs were conducted in the vegetative period in a laboratory using water samples from a reservoir. The samples have been taken during field trips around a pond.

Species composition of the phytoplankton has been established from the sample through the method of separation on filters and consequent observation through microscope. Then the sample has been poured into two round 250 ml flasks. One flask was experimental, the other control. Nutrients were added into the experimental flask at a ratio N:P=8, in which the green-blue algae have dominant position in the phytoplankton environment (Schindler, 1977. Quoted from Levich et al., 1977). The samples were cultivated under illumination with Osram Plantastar 250 W lamps in temperatures of 20-24° C.

After 6 to 9 days different species of algae propagated within the samples and water in the flasks started to bloom. Species composition of the phytoplankton has been determined in both flasks and compared with the original composition.

Descriptions of individual experiments with water samples collected in different parts of the pond are listed bellow.

### Experiment No. 1

Sample has been collected in one part of the pond. During observation of the sample through microscope the following algae strains have been detected:

| | |
|---|---|
| 1. | Cyclotella comta |
| 2. | Opephora Martyi |
| 3. | Synedra ulna |
| 4. | Navicula pupula |
| 5. | Navicula pupula |
| 6. | Navicula dicephala |
| 7. | Navicula exigua |
| 8. | Gomphonema olivaceum |
| 9. | Melosira granulata |
| 10. | Surirella robusta |
| 11. | Nitzschia sublinearis |
| 12. | Scenedesmus quadricauda |
| 13. | Scenedesmus q. f. setosus |
| 14. | Chlorella vulgaris |
| 15. | Gigantochloris permaxima |
| 16. | Oocystis solitaria |

The samples have been separated into two 250 ml flasks and cultivated according to the above described procedure.

On the 12^{th} day of cultivation uniform green coloration has been observed in the experimental flask. Blue-green fouling appeared in the control flask. During direct microscopic observation of the preparations the following algae strains have been detected:

| Experimental | Control |
|---|---|
| 1. Navicula exigua | 1. Anabaena variabilis |
| 2. Navicula lanceolata | 2. Anabaena constricta |
| 3. Cocconeis pediculus | 3. Merismopedia glauca |
| 4. Chlorella vulgaris | 4. Synedra ulna |
| 5. Scenedesmus quadricauda | 5. Synedra ulna v. danica |
| 6. Scenedesmus acuminatus | 6. Synedra acus |
| 7. Scenedesmus q. f. setosus | 7. Synedra amphyrinchus |
| 8. Oocystis natans | 8. Navicula exigua |
| 9. Pandorina morum | 9. Navicula lanceolata |
| 10. Ankistrodesmus falcatus | 10. Navicula placentula |
| 11. Golenkinia radiata | 11. Navicula pupula |
| 12. Cystodinium Steinii | 12. Cyclotella comta |
| | 13. Cocconeis placentula |
| | 14. Opephora Martyi |
| | 15. Scenedesmus q. f. setosus |
| | 16. Scenedesmus quadricauda |
| | 17. Scenedesmus acuminatus |
| | 18. Pandorina morum |
| | 19. Kirchneriella lunaris |
| | 20. Chlorella vulgaris |
| | 21. Ankistrodesmus falcatus |
| | 22. Pediastrum tetras |

The contents of the control flask, after thorough stiffing, have been uniformly divided into two flasks. Equal amount of suspension from the experimental flask has been added into one of them. The cultivation of the combined (experimental+control) and control samples continued under the same lighting and temperature conditions.

On the 20^{th} day of growing a visual inspection of the experimental flask (experimental+control) discovered a uniform intensive green coloration. Impure green sediment has been discovered on the bottom of the flask. The water in the control flask turned transparent with numerous blue-green aggregates, either floating or deposited at the bottom. During direct microscopic observation of the preparations the following algae strains have been detected:

| Experimental + Control | Control |
|---|---|
| 1. Synedra ulna v. danica | 1. Anabaena variabilis |
| 2. Synedra ulna | 2. Anabaena constricta |
| 3. Synedra Vaucheriae | 3. Anabaena aequalis |
| 4. Navicula exigua | 4. Merismopedia punctata |
| 5. Nitzschia palea | 5. Merismopedia glauca |
| 6. Rhizosolenia longiseta | 6. Synedra ulna |
| 7. Golenkinia radiata | 7. Synedra ulna v. danica |
| 8. Pediastrum tetras | 8. Synedra amphyrinchus |
| 9. Oocystis natans | 9. Navicula exigua |
| 10. Pandorina morum | 10. Navicula lanceolata |
| 11. Chlorella vulgaris | 11. Navicula placentula |
| 12. Scenedesmus acuminatus | 12. Nitzschia palea |
| 13. Scenedesmus quadricauda | 13. Pandorina morum |
| 14. Scenedesmus q. f. setosus | 14. Scenedesmus quadricauda |
| 15. Ankistrodesmus falcatus | 15. Scenedesmus acuminatus |
| 16. Cystodinium Steinii | 16. Scenedesmus q. f. setosus |
| | 17. Cystodinium Steinii |

Volume of raw biomass has been detected in the output samples by weighing on analytic balance.

Experimental+Control

237 ml of the sample contained 0.092 g of raw biomass, in 11-0.388 g respectively, 1 m³-388 g, 1 ha of water surface in 1 m water column-3.88 t.

Control

100 ml of the sample contained 2.384 g of raw biomass, in 11-23.84 g respectively, 1 m³-23.84 kg, 1 ha of water surface in 1 m water column-238.4 t.

In the result of the experiment the water bloom cells decayed (lysis) due to the effect of blooming green algae. The green algae subdued the propagation of blue-green algae regardless of the fact that volume of the blue-green algae biomass exceeded the green algae biomass more than 50 times.

### Experiment No. 2

Sample has been taken from a natural watershed. During direct microscopic observation of the preparations the following algae strains have been detected:

```
  1. Anabaena constricta
  3. Synedra ulna
  4. Synedra ulna v. danica
  5. Synedra ulna
  6. Synedra acus
  7. Fragilaria capucina
  8. Opephora Martyi
  9. Scenedesmus acuminatus
10. Cymbella ventricosa
11. Navicula placentula
12. Navicula laterostrata
```

According to the above mentioned procedure the sample has been divided into two flasks—experimental and control. The cultivation took place under identical conditions. Virescence has been detected in the experimental flask on day 6. The control flask, however, contained a pale green sample with thread-like elements and blue-green sediment. During direct microscopic observation of the preparations the following algae strains have been detected:

| Experimental | Control |
|---|---|
| Synedra ulna | Oscillatoria sancta |
| Synedra ulna v. danica | Anabaena constricta |
| Scenedesmus quadricauda | Cymbella affinis |
| Scenedesmus q. f. setosus | Synedra ulna |
| Oocystis natans | Synedra ulna |
| Oocystis pelagica | Synedra acus |
| Chlorella vulgaris | Fragilaria capucina |
| Tetrastrum multisetum | Fragilaria crotonensis |
| Golenkinia radiata | Cyclotella comta |
| Cystodinium Steinii | Navicula exigua |

The bloom in the experimental flask has been caused by Scenedesmus quadricauda and Oocystis pelagica. Threads and blue-green sediments in the control flask were mostly represented by Anabaena constricta.

The experimental flask did not contain water bloom due to intensive propagation of green algae of the bloom.

### Experiment No. 3

Sample has been collected in the different part of the pond. During direct microscopic observation of the preparations the following algae strains have been detected:

```
  1. Anabaena variabilis
  2. Oscillatoria sancta
  3. Cyclotella comta
  4. Melosira granulata
  5. Opephora Martyi
  6. Chlorella vulgaris
  7. Scenedesmus quadricauda
  8. Scenedesmus q. f. setosus
  9. Scenedesmus acuminatus
10. Kirchneriella lunaris
11. Gigantochloris permaxima
12. Coelastrum microporum
13. Pandorina morum
14. Crucigenia rectangularis
15. Ceratium hirundinella
16. Menoidium falcatum
17. Phacus longicauda
```

On the 9^{th} day of cultivation a green coloration of the sample has been observed in the experimental flask. The color of the control flask sample has not changed. During direct microscopic observation of the preparations the following algae strains have been detected:

| Experimental | Control |
|---|---|
| Synedra ulna | Anabaena variabilis |
| Navicula exigua | Synedra ulna |
| Nitzschia communis | Synedra ulna v. danica |
| Oocystis pelagica | Rhizosolenia longiseta |
| Oocystis natans | Pediastrum boryanum |
| Chlorella vulgaris | Pediastrum sp. |
| Scenedesmus quadricauda | Cymbella tumida |
| Scenedesmus q. f. setosus | Nitzschia communis |
| Scenedesmus acuminatus | Oocystis natans |
| Kirchneriella lunaris | Scenedesmus quadricauda |
| Golenkinia radiata | Scenedesmus acuminatus |
| Coelastrum microporum | Coelastrum microporum |
| Tetraedron Schmidlei | |
| Chlamydomonas sp. | |

As the list indicates Anabaena variabilis did not propagate in the experimental sample, probably due to intensively growing green algae. This algae has been, however, present in the control as well as in the original water sample.

### Experiment No. 4

Sample has been taken from the second pond. The experiment was carried out according to the different procedure. For the purpose of assessment of pure chlorella effect on the species composition of the phytoplankton 10 ml of chlorella suspension, with density of 1 million cells per milliliter, have been added into the experimental flask. 100 ml from the experimental flask from 11.06.2003 have been added to the water sample from the pond in the second experimental flask. The algae species composition in the experimental flask from 11.06.2003 was the following:

```
  1. Navicula dicephala
  2. Chlorella vulgaris
  3. Scenedesmus quadricauda
  4. Scenedesmus q. f. setosus
  5. Scenedesmus acuminatus
  6. Oocystis natans
  7. Kirchneriella lunaris
  8. Lagercheimia genevensis
  9. Golenkinia radiata
10. Coelastrum microporum
11. Cystodinium Steinii
12. Ankistrodesmus falcatus
```

After 9 days of growth the chlorella culture suppressed the growth of virtually all algae, including three Anabaena species. Only smaller growth of Oscillatoria sancta has been detected in the chlorella culture. In the experimental flask (sample+experimental) blue-green algae did not grow at all.

Thus, the chlorella strain together with green algae suppressed the propagation of blue-green algae contained in the original as well as in the control sample.

A watershed is being inoculated by 20 liters of Parachlorella KIEG 1904 suspension with density of 60 million cells per milliliter in March. This procedure helps to prevent growth of water bloom during summer

4. Water treatment

The table below contains data showing the effects of chlorella on water for the purposes of water softening (water softens with changing of hydrogen ion exponent (pH)).

TABLE

| % of algae | | | |
|---|---|---|---|
| suspension | pH | KH | GH |
| 0 | 7.6 | 20°d | >21°d |
| 10 | 7.6 | 15°d | >21°d |
| 20 | 8 | 15°d | >21°d |
| 30 | 8 | 15°d | >21°d |
| 40 | 8 | 15°d | >21°d |
| 50 | 8 | 10°d | >21°d |
| 60 | 8 | 10°d | >21°d |
| 70 | 8 | 6°d | >21°d |
| 80 | 8 | 6°d | >21°d |
| 90 | 8.4 | 6°d | >21°d |
| 100 | 9 | 6°d | >21°d |

At the beginning of the experiment, with zero concentration of the Parachlorella KIEG 1904 suspension, the value of carbonate hardness kH equals to 20 units in German degrees. Carbonate hardness of water gradually decreases with the strain growth, and after reaching the level of saturation (algae growth stops) the water hardness equals to 6 degrees and pH-9.
5. The method of organic waste processing starts with all organic waste being fed into the module 1. Separation of glass and metal will take place there, as well as mincing and stabilization of food remains and other waste. Stabilization takes place through supply of air aerobic bacterium. The waste is fed through a biotunnel, where the temperature is raised and water evaporates, which is further been condensed and utilized for other purposes. After the waste passes the biotunnel, it is compressed into pellets, which are transported by carbon dioxide into the loading device, from which they are transferred by an auger into dispenser 2.3, where the waste is compressed again. Then it is discharged by carbon dioxide into the reactor 2.1. This reactor contains melt of various salts with temperature from 900 to 1000° C. Pyrolysis occurs in the reactor 2.1, releasing particular elements C, H, N, S, O2, and methane and other gases. The composition of gases is given by the pressure in the thermal cracker. The methane volume is modified by external pressure regulation. Metal oxides, which are contained in the pyrolysis waste, are reduced to pure metals using a melt. The top part of the reactor 2.1 contains gases and the bottom part—carbon and pure metals. The level of melt is also regulated through pressure, while being mechanically stirred. After conclusion of thermal cracking, while the cracking time is determined by a calculation, valve 2.6 connecting the pipes opens, and the melt is transferred under pressure into the reactor 2.2. The reactor 2.1 is connected by two pipelines with gasification reactor 2.2. Gases from the top part of the reactor 2.1 are transferred into the bottom part of the reactor 2.2 through pipes 2.6.1. The melt from the reactor 2.1 goes through pipes 2.6 into the bottom part of the reactor 2.2. Steam from the steam generator 2.10 is being fed into the top pipe 2.6.1. Gasification occurs in the reactor 2.2 in the presence of steam, under temperatures 940-1000° C., while H, CO, CO2, H2S, NH4, CH4 and other hydrocarbons are created, as per presence of elements. The volume of methane and other hydrocarbons, up to butane, is controlled by the means of pressure. The gasses pass into the upper part of the reactor's chamber 2.2 and stream through pipes 2.6.2 runs into the cyclone separator 2.5, where it is been mechanically purified. Mechanic particles return into the feeder 2.3. Pure gas streams through pipes 2.6.3 into the feeder 2.8, where it is analyzed for presence of dangerous gases FH or dioxins. In case dangerous gases are detected the valve on the pipe 2.6.5 closes and valve on pipe 2.6.4 opens. The contaminated gas returns into the reactor 2.1, waste feed from the feeder 2.3 is cut and hydroxides are fed from the feeder of elements for gas quality correction 2.11 are added into the feeder 2.3 for neutralization. Then the process repeats. When gasification in 2.2 is concluded, manifold 2.6.6 valve opens and part of the increased level of melt will automatically pass into the filtration device 2.4 containing zirconium filter with apertures of different diameters, through which the melt passes and where the metals are captured. Saturated filter is placed into an induction furnace 10, where the filter is pulverized in an inert environment and the powder is used as a raw material for further processing. The melt passes from the device 2.4 through manifold 2.6.7 into the pump chamber 2.9, from which it is pushed though manifold into the reactor 2.1. This completes the cycle. Sewage, also considered a source of organic waste, is transferred into a reservoir 11, where it is been oxidized using anaerobic bacterium, producing sludge and technical water. The sludge can be mixed into the liquid waste reservoir 2.3 either alone, or together with solid waste.

### Module 1

Solid household (industrial, municipal, alimentary) waste will be transported to module 1 for waste preparation. Primary waste separation takes place in this module. Ferrous and nonferrous metals and glass are removed during separation, and primary waste pulverization takes place. After separation and pulverization the waste is transferred into a biotunnel. Biotunnel is a facility with pressure lower than the atmospheric—183 mm of mercury column—and with organized system of air supply through waste and sewage of liquid products, watered by the amount of waste (contaminated water). Anaerobic microbes, present in food-stuff, process alimental organic matter during passage through the exhaust of oxygen, present in air, radiate heat and increase the temperature of the entire volume of waste. During this process and under the decreased pressure in the biotunnel water evaporates from the waste at 65° C. and food-stuff waste stabilizes. Stabilized and dried waste passes to repeated pulverization, compaction and molding through dies. Waste processed by this method either passes further into special warehouses or is transported by gas to module 11—module for preparation and drying of active sludge, separated from industrial and household sewage—and mixing of prepared waste in module 1 with pyrocarbon, passing from module 2, and crystallized salt particles. Carbon dioxide serves as the transport gas. Contaminated water from the biotunnel system passes into module 11.

### Module 11

Module 11 comprises of interconnected sewage purification block and block for mixing of prepared waste and sludge drying. The sewage purification block comprises of reactors connected in succession. The technology of the block operates by passing sewage water, which spills from the first to the last reactor, while in each reactor in undergoes a full biological purification cycle. The active sludge, passing between reactors, is separated into particular flows. Therefore the block prevents discharge of sewage water with increased concentrations of toxic contamination to active sludge (SPAN, chlorine, manganese etc.). This enables its utilization for complex biological treatment of sewage water (industrial-household, rainwater and water containing oil products). Excess active sludge in module is automatically discharged and is passed to drying and than to mixing of prepared waste in module 1 with waste passing from module 2 with pyrocarbon and crystallized salts. Process water, produced in the module, is further utilized in operation of the entire facility. In case of absence of waste water supply into the module the wastes, passing from the module 1 and module 2 are only mixed, and transported by gas into the module 2.

### Module 2

Prepared waste from module 11 is transported by transport gas into the receiving chamber 2.3 of the module 2. Transfer into the receiving chamber is also possible through slide valve g-01 for bulk materials from block 2.11—block for storage of elements correlating with qualitative composition of gas from module 2.8. End of transport of waste from module 11 is determined by filling the receiving chamber 2.3. From the receiving chamber 2.3 the waste is transported using the auger 2.3.0 into the chamber for waste batch molding 2.3.1. Compaction takes place in the chamber, forming a gas-solid plug, loosening and removal of the upper part of the plug back into the receiving chamber. The plug is than shot back into the salt melt, which is contained in the reactor for pyrolysis 2.1. The melt of specially selected salts is maintained at 940-1040° C. The waste, which passed into the melt, is subjected to pyrolysis and partial gasification due to water contained in the waste and due to recreated pyrogenation water. The gases from pyrolysis and gasification, flushed through the melt content, are contained in the gas chamber of the pyrolysis reactor. Special mechanical devices in the reactor for pyrolysis 2.1 perform mixing of melt and contained gases and solid elements. Heating elements, placed in the reactor for pyrolysis 2.1, provide source of heat to maintain temperature in the melt. After reaching the necessary pressure within the reactor valves v-01 and valve v-11 automatically open and the gaseous content from the reactor for pyrolysis 2.1 is passed though manifold 2.6.8 into the gasification reactor 2.2 beneath the analogous salt melt and the melt content passes through manifold 2.6.1 from the reactor for pyrolysis 2.1 into the gasification reactor 2.2. After pressure equalization in the reactor for pyrolysis 2.1 and gasification reactor 2.2, valves v-01 and v-11 close. The melt content, transferred from the reactor for pyrolysis 2.1 into the gasification reactor 2.2, is replaced by purified content of salt melts from the pump chamber of the purified melt 2.9 by supplying transport gas to the pump chamber piston 2.9 and automatic opening of valve v-08. After transferring the products from the reactor for pyrolysis 2.1 into the gasification reactor, water steam is supplied to facilitate gasification of pyrocarbon. Special mechanic devices in the gasification reactor 2.2 facilitate mixing of melt and gases contained in it, water vapors and pyrocarbon, guarantees presence in the melt for more than two seconds. Heating elements, placed in the gasification reactor 2.2, provide source of heat to maintain temperature in the melt. After reaching the prescribed maximum operating pressure the valve v-12 in the gasification reactor opens automatically to facilitate transfer of generated gases into the device for purification of gas from solid particles. Presence of solid particles in the gas is caused by separation of salt and pyrocarbon particles by gas, which didn't react in the gasification process. When the pressure in the gasification reactor falls to the minimal operating pressure, valve v-12 closes and v-02 opens. The melt content, transferred from the reactor for pyrolysis 2.1 through manifold 2.6.2, is further passed into the block 2.4—device for filtration of metal from melts, recovered in environment containing hydrogen and pyrocarbon. In course of the pyrolysis and gasification process metal oxides, contained in waste (determining such factors as ash content) at temperatures exceeding 500° C., are recovered into pure metals. In block 2A replaceable zirconium filters with branched mosaic surface, facilitating filtration of recovered metals particles from salt melt. The filters are replaced when they are filled by metal particles. The purified batch of melt is heated by heating elements to temperatures of 940-1040° C. and passed into the pumping chamber of the block 2.9, and as described above, it returns into the chamber of the reactor for pyrolysis 2.1. Replaceable filters are installed into the inert gas environment and, after pulverization, can become a raw material for metal smelting in the induction furnace 10.

Pyrocarbon and melt salts return after purification into the module 11 to be mixed with the main flow of waste and for reprocessing in module 2. Purified gas passes from the device for gas purification into the damping reservoir 2.8 for analysis of qualitative composition of the product gas. In the event of detection of gases containing HCl and HF, the transfer of gas into the module for gas synthesis preparation is terminated by automatic closure of valve v-10 and opening of valve v-09. “Contaminated gas” from module 2.8 is passed into the reactor for pyrolysis 2.1. Elements, which will neutralize detected HCl and HF gases in the process of pyrolysis and gasification, start to pass into the receiving chamber 2.3 through slide valve g-01. When quality factors of the gas in module 2.8 are recovered, valve v-09 closes and valve v-10 opens, and gas, containing CO2, CO, H2, CH4 and its homologues up to C4, passes into the module 3 for synthesis gas preparation.

In case of utilization of liquid hydrocarbon material, oil sludge, fuel oil, glycerin or extraneous oil gases, natural gas, during the supply into the reactor for pyrolysis the filling device 2.3 is replaced by block 2.3.3 for organization of supply of the above raw materials. Because the side product in the process of esters production of complex methyl ester acids in the module 9 is technical glycerin, it will be utilized in the module as a source of H2 and CO. Glycerin formula —C₃H₅(OH)₃. Its molecule comprises of 3 carbon atoms, 3 oxygen and 8 hydrogen atoms. In case hydrogen is generated, it will become a rich fuel source with renewable resources.

### Module 3

Performance of the module 3 is based on the function of synthetic polymer membranes—thin polymer membranes, which act as selective barriers for gaseous mixtures even during mixing of necessary gases in respect of its purpose. There are two existing types of membranes: semi-fibrous and flat membranes. Semi-fibrous membrane comprises of porous polymer fiber with gas separating layer, deposited on its outer surface, with thickness of 0.1 μm max, ensuring high gas permeability. Porous fiber has complex asymmetric structure of a pad; the density of the polymer increases in relation to proximity to the outer surface of the fiber, enabling gas separation under high pressure. The separation of the gas mixture takes place due to the difference of partial pressures on the outer and inner surfaces of the membrane, tightly packed in the special-design membrane cartridge, into which the raw gas is pumped under pressure. Gases, slowly permeating through the membrane (e.g. CO, N₂, and CH₄) pass out of the membrane module through a discharge spout. Gases, permeating through polymer membrane quickly (e.g. H₂, CO₂, O₂), pass into the fibers and from the membrane cartridge through second discharge spout for further utilization.

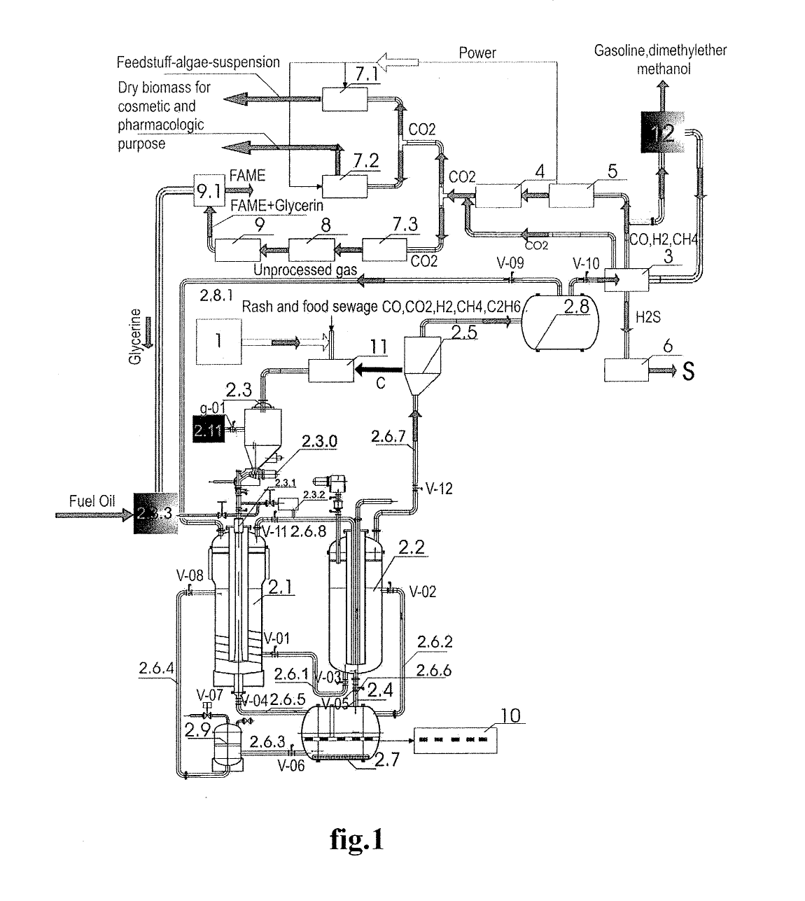

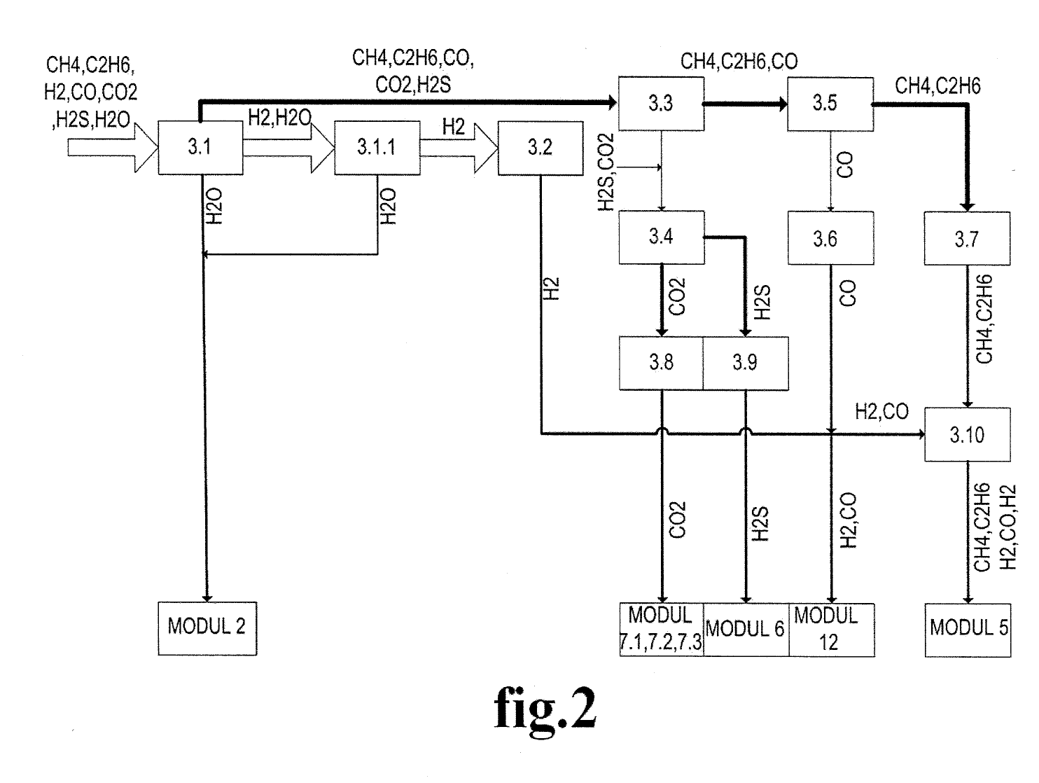

The gas, purified from mechanical ingredients, passes into:
Waste pyrolysis and gasification module 2,
Methanol or dimethylether or high-octane gasoline production module and into block 3.1, FIG. 2, where hydrogen and moisture are removed from the supplied gas on a selected membrane. Separated H₂ from the first discharge spout of the membrane 1 passes into the drying block 3.1.1 and further into the gas tank 3.2 for storage. The flow of the remaining gas, comprising of CO₂, NH₃, CO₂, CO, CH₄, C₂H₆ . . . , from the second discharge spout of membrane 1 runs into block 3.3, where priority removal of hydrogen monosulphide and carbon dioxide takes place on a selected membrane. The produced mixtures of H2S and CO2 from the first discharge spout of the membrane 2 passes into the gas tank 3.4 for temporary storage. The flow of the remaining gas (CO, CH₄, C₂H₆,) from the second discharge spout of the membrane 2 passes into the block 3.5, where a priority separation of carbon oxide from the remaining gas takes place on a selected membrane. From the first discharge spout of the membrane 3 the separated CO gas passes into the gas tank 3.6 for storage. The flow of the remaining gas CH₄, C₂H₆ passes from the second discharge spout of the membrane 3 into the storage tank 3.7 for storage.

The mixture of gases H2S and CO2 from the gas tank 3.4 passes to membrane 4 specially manufactured on a porous polymer composite backing, which includes fluorine-containing polymer with a modifier, and drying, by the use of copolymer trifluorethylen with vinilidenfluorid as fluorine-containing polymer and perfluorcarbon lubrication KC as modifier, representing 3.10% of the total polymer volume. After drying the membrane is processed with plasma from a glow tube. Important feature of the membranes, manufactured in accordance with this invention, is the fact, that they block H₂S and let CO₂ through, while in known technical solutions H₂S passes through faster than CO₂. Resulting “turn” in selectivity in the membrane material, as compared with the previously offered, significantly enhances possibilities of design of membrane apparatuses. “Enrichment” of the mixture by the H₂S component is especially important for sulfur production. The separated CO₂ gas passes from the first discharge spout of the membrane 4 into the gas tank for storage. The flow of the remaining H₂S gas from the second discharge spout of the membrane 4 passes into the gas tank 3.9 for storage and further into the module 6 for elemental sulfur production. Combustion gases H₂, CO, CH₄, C₂H₆ are mixed in block 3.10 in respect of further utilization of these products:
Complete utilization of a mixture of combustion gases CO, CH₄, C₂H₆ . . . , H₂ for heat and power generation, for internal consumption in the facility as well as for sale, its supply to the heat and power generation module 5,
Complete utilization of gases CH₄, C₂H₆ . . . and partial utilization of CO, H₂ for heat and power generation, for internal consumption in the facility, passing to the module 5, and remains of CO, H₂ in a ratio necessary for the synthesis of methanol, dimethylether or high-octane gasoline, passing into the module 12.
   The produced gas CO₂ runs from the gas tank 3.8 to modules 7.1, 7.2, 7.3 as a raw material for biological synthesis.

### Module 5

The mixture of gases CH₄, C₂H₆ . . . , H₂, CO from the block 3.10 of the module 3 passes into the combustion turbine of the module 5.

Multistage compressor in the combustion turbine compresses the atmospheric air and passes it under high pressure into the combustion chamber. Certain amount of fuel also passes into the combustion chamber of the combustion turbine. Fuel and air upon collision at high speed ignites. The fuel-air mixture burns, producing large amount of energy. Then the energy of combustion products transforms in the combustion chambers into mechanical work through turning the turbine blades by gas jet.

Certain part of the generated energy is consumed during air compression in the compressor. The remaining part of work is transferred to the power generator. The work consumed by this aggregate is an effective work of the combustion turbine. Combustion gases containing high levels of CO₂ pass through the rendering plant to generate thermal energy, and then they run into the module for smoke gases recovery, module 4.

Combustion turbines may be fitted with heat utilization system. This means that module 5 can be used as a thermal power plant.

### Module 4

Carbon dioxide is recovered from combustion gases, transferred from module 5. Exhaust combustion gases, containing nitrogen, water vapors and carbon dioxide and also micro-additives, pass for heating to the evaporator of water steam generator, and then, through the heat exchanger, into the gas-separating membrane device. “Dry” water vapors out of the evaporator equalize the temperature with exhaust gases in the heat exchanger under atmospheric pressure, and pass into the cavity below the membrane of the gas separator, while it creates conditions of decreased partial pressure of gasses, including carbon dioxide. Carbon dioxide from the exhaust gases in the membrane device is absorbed on the surface of chemically active, e.g. amino-containing membrane (NH₂-amin): CO₂+2RNH₂═(RNH₂)2CO₂, where R is the immobilizing chemical group of the polymer, which facilitates mobility of carbamate (RNH₂)₂CO₂ in the membrane. On the exhaust side of the membrane, due to decreased partial pressure in the water vapor environment within the cavity below the membrane, the carbamate ion (RNH₂)₂ CO₂ breaks down discharging CO₂. Gases chemically inert to the group of amines—nitrogen, oxygen—practically never permeate the membrane, due to the membrane discrimination according to CO₂ at least 100 and concentration of carbon dioxide up to 92 99 for the input concentration from 10 to 60. Water vapor is passed through gas separator with minimal consumption of 2.3 mCO₂, where m=molar consumption of CO₂ permeating through membranes. The produced gas-vapor mixture “CO₂+H₂O” is collected in the tank, through exchange of contents and condensation of water vapors under stable total pressure, which equals the atmospheric, and temperature, set accordingly in the range from 354 to 363 K (condensate). Then the produced gas runs through drying and collection of pure CO₂ in gas tank. There is a possibility of gas compression into liquid phase to decrease the contents of storage tanks. The CO₂ gas, produced in the module 4, passes to modules 7.1, 7.2, 7.3.

### Module 7.1

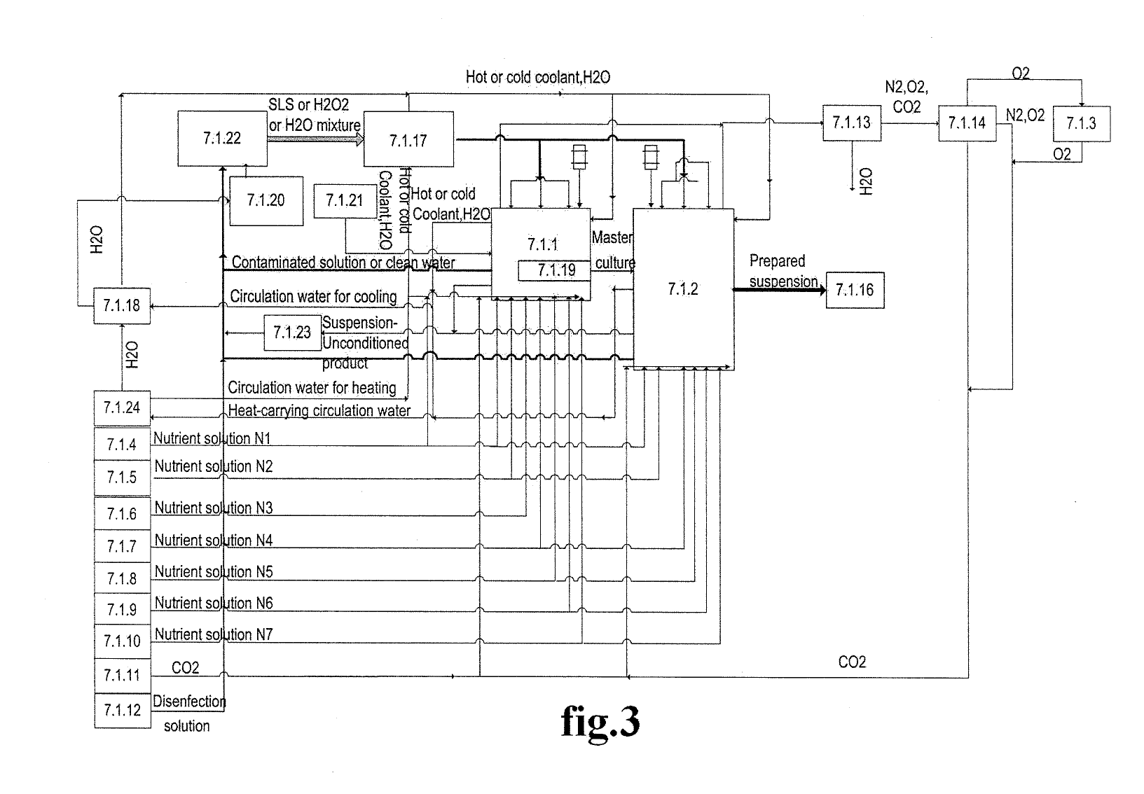

Module 7.1 is the basic block for modules 7.2 and 7.3. Module 7.1 comprises of, according to FIG. 3:
Photobioreactor of mother solution—7.1.1;
Photobioreactor of operational culture—7.1.2;
Suspension oxygenator—7.1.3;
Unit for preparation and nutrition supply N 1-7.1.4;
Unit for preparation and nutrition supply N 2-7.1.5;
Unit for preparation and nutrition supply N 3-7.1.6;
Unit for preparation and nutrition supply N 4-7.1.7;
Unit for preparation and nutrition supply N 5-7.1.8;
Unit for preparation and nutrition supply N 6-7.1.9;
Unit for preparation and nutrition supply N 7-7.1.10;
Unit for preparation and supply of CO2 N 1-7.1.11;
Unit for preparation and supply of disinfecting solution N 1-7.1.12;
Unit for degassing of CO2 and O2 from water solution of the suspension—7.1.13
Unit of technologies of the adsorption short cycle (KCA) and vacuum short cycle (VKCA) for separation of N₂, CO₂ and O₂, including the station for filling oxygen bottles—7.1.14;
Unit for storage of the finished suspension of Parachlorella KIEG 1904—7.1.16;
Unit for heating of the suspension and original water—7.1.17;
Unit for suspension cooling—7.1.18;
Unit for supply of the mother solution into the photobioreactor's operational culture—7.1.19;
Unit for original water treatment—7.1.20;
Unit for preparation and supply of basic strain to the mother solution's photobioreactor—7.1.21;
Unit for preparation and supply of disinfecting solution—7.1.22;
Unit for temporary storage of unconditioned product—7.1.23;
Unit for reception of water from source—7.1.24

The photobioreactors for mother solution 7.1.1 and working solution 7.1.2 are of the same design. The difference is in the volume of reactors. The reactor's design represents vertical tank with a removable top lid. There is a shaft installed along the reactor's axis with an attached sectional facility (split into segments). The segments are isolated from each other by side plates and have open top and bottom space. Lights, based on LED RGB matrix, in underwater configuration are mounted on side plates within segments to supply light of the required wavelength. The number of lamps depends on the segment's volume and radiant density to facilitate the effective photosynthesis. The shaft is mounted in sliding bearings on the top and bottom lids of the reactor and can be turned over due to supply from the electric motor and reducer. Sprinkling heads are mounted in the top part of the reactor and supply liquid or air under pressure for disinfection or purging the structure and shell of the reactor of stuck microorganisms. A rack made of perforated tubes is mounted in the bottom part below the structured device; the tubes are connected with outer sockets for input of nutrition and pure CO2 or CO2 solution, air or oxygen. The process starts at the moment of raising the mother solution of Parachlorella KIEG 1904 in the mother solution's photobioreactor 7.1.1; it continues by passing the necessary volume of the mother solution into the processing photobioreactor 7.1.2, facilitation of lag phase of the mother solution growth, and ends with daily extraction of a part of the culture to the storage block and shipping the product to customers. Disinfection, consisting of two operations, takes place prior to placement of the mother solution into the photobioreactor of mother solution and working photobioreactor:
Washing of the bioreactor and equipment with soap solution SLS at 80° C., followed by flushing of the system,
Washing of the bioreactor and equipment with H₂O₂ solution and consequential flushing of the system.

### Disinfection of the System

Selected type of disinfecting solution, prepared out of water supplied to the unit for water reception from source 7.1.24 and disinfection components, from the unit for preparation and supply of disinfecting solution 7.1.22 is being pumped through the unit for heating of the suspension and original water 7.1.17. Then it runs into the solution supply circuit, from which is passes to the sprinkling heads, which create curtain of solution sprayed under pressure over the entire cross-section area of the reactor. Solution, collected in the bottom part of the reactor, is pumped into the unit for preparation and supply of disinfecting solution 7.1.22, where it is heated and returned into the reactor for spraying. Cycles are repeated until the end of specified period of disinfection. Contaminated disinfecting solution is discharged into the industrial sewage, and clean water for flushing is prepared in the block 7.1.22. Cycles and supply of clean water for flushing are similar to those for disinfecting solution. After specified time elapses the flushing is repeated according to set number.

### Growth of Mother Solution

Water runs from the source into the unit for heating of the suspension and original water 7.1.17 and is heated to a set temperature depending on the selected process mode, i.e. culture growing or disinfection, and further passes into the photobioreactor of mother solution. After reaching the operational level in the photobioreactor of mother solution the system maintains initial level of dissolved CO₂ and O₂ and also pH of original water. Based on data from original water pH value analysis and basic ions Ca+ and Mg+ contained in it, the process control system calculates balanced ratios in the system with the given amount of dissolved carbonate components at the measured pH value, i.e. amount of residual carbonates and produced bicarbonates prior to supply of original water into the mother solution's reactor. Then water supplied into the mother solution's bioreactor is heated to set temperature by supply of heating media into the photobioreactor's shell. The heating medium—hot water—is heated in the water circulation circuit—hot heating medium tank—pump—heat exchanger with electric heating—shell of the mother solution's bioreactor—hot heating medium tank. After reaching the specified temperature the system for automatic maintenance of the bioreactor's temperature and specified frequency of rotation of the sectional device switches on. The temperature is regulated by hot or cold heating medium supplied into the bioreactor's shell. Coolant moves through water circulation circuit—coolant tank—pump—module cooling tower—mother solution's bioreactor shell—coolant tank. When specified temperature in the mother solution's photobioreactor is reached, the control system maintains the value of CO₂ and O₂ dissolved in water and also pH value of the prepared water. Based calculated original water pH value and remaining basic ions Ca+ and Mg+, the process control system calculates balanced ratios in the system with the given amount of dissolved carbonate components at the measured pH value, i.e. amount of residual carbonates and produced bicarbonates after heating of original water in the mother solution's reactor. The algorithm repeats with respect to constantly changing balance of reactions.

CO2+H2O< >H2CO3< >[H+][HCO3−]+CaCO3< >Ca(HCO3)2

CO2+H2O< >H2CO3[H+][HCO3−]+MgCO3< >Mg(HCO3)2

The algorithm enables calculation of carbonate hardness and CO₂ supply after measuring the values of O₂ and CO₂ dissolved in water, as one of sources for photosynthesis, with the object of material balance calculation of the photosynthesis process itself.

After setting the temperature and frequency of rotation of the sectional device in the mother solution's bioreactor the control system maintains these parameters in an automatic mode. Nutrition solutions of microelements, which will determine the features of the future product—according to its purpose—feedstuff, dry biomass for pharmaceutical and cosmetic industry, or methylester production—will be supplied from the supply unit 7.1.4-7.1.10 through sockets connected with the supply rack by dosing pumps. The complex of sensors, placed in the mother solution's bioreactor, enables measurement of quantitative values of microelement ions, which originated in the water suspension, and also their change in the course of biological processes.

Time is set in the control system “operator—machine” that determines the period of culture's introduction into the content of the mother solution's bioreactor or culture awakening. Certain period is associated with biologic clock of the culture, during which it can be put out to the prepared environment for further growth. Duration of this period from the beginning is one hour. If the operator initially issues a command for inputting the culture into the photobioreactor, where the control system is able to determine the presence of culture in the reactor by its optical density as compared to the optical density of the prepared original water supplied after nutrition, the mother solution in the amount specified by the operator from the unit for preparation and supply of basic strain 7.1.21 is transferred into the mother solution's photobioreactor. If the culture has already been input into the mother solution's photobioreactor, this stage is skipped.

The control system in each segment then fluently sets the required radiant density of the light flux and frequency of radiation for the period specified by the culture's introduction into the volume of the mother solution's bioreactor. In case of need it is possible to obtain the light flux with radiation in red, blue, green, white or other range from 400 to 700 nm. Differentiation based on frequency and intensity of the light flux is possible in every individual segment of the device.

Concurrently with the given process the control system initiates the CO₂ supply from the CO₂ source.

### CO₂ Sources

Source of CO₂ can be a gas or liquid product in pure form or water solution of CO2 produced by special fermentation processes. Based on temperature data in the bioreactor the system reports to the operator possible amount of CO₂, which can be dissolved in water, and concentration of CO₂ actually dissolved in water. Gaseous CO₂ from special containers, from the CO₂ preparation and supply unit N 1 7.1.11, is fed into the mother solution's photobioreactor through gas consumption batch meter. Liquid CO₂ evaporates and is then supplied into the volume of mother solution's photobioreactor. The method of preparation of CO₂ solution is described below. Straw dried molded pellets are filled in a special container, with built-in tank-filter, in the CO₂ preparation and supply N 1 7.1.11. Water with temperature of 29 to 37° C. is poured over the pellets. Important feature is that the molded and dried pellets do not contain or contain very little amount of oxygen. Aerobian cellulose destroying Vibrio Vulgaris type bacteria and also bacteria of the Sorangium and Cytophaga families are introduced into the environment together with the pellets. The optimal growing conditions are pH=7.0 and temperature 37° C. In the process of cultivation the following substances are produced together with cellulose: H₂, CO₂, acetic acid and reducing sugars, especially glucose and cellobiose. The bacteria do not use glucose, i.e. in the CO₂ preparation and supply unit's tank water contacts with pellets, and the aerobian bacteria decompose cellulose creating monosaccharides: glucose, fructose and cellobiose. Part of the sugar is oxygenated by oxygen and been dissolved in original water producing carbon dioxide, e.g. C6H12O6c=6CO2oo+6H2O. The c—index shows that glucose is originated from cellulose; oo—index shows that CO₂ is created by oxygenation of an organic matter. Solution created by this method contains the initial value of CO₂ concentration in the solution and certain concentration of monosaccharides.

After the introduction of gaseous CO2 into the mother solution's reactor, dissolution of it or monosaccharides solution with the initial CO2 content, there is a balance created in the photobioreactor system between the dissolved CO2, dissolved monosaccharides, carbonates and bicarbonates that can accumulate CO2, and the dissolved oxygen, initial concentration of which is determined by original water. For the radiant density a nutrients' concentration required for the given moment the culture is prepared to start the process of photosynthesis. If the initial concentration of CO₂ (soft water) and O₂ is low, and monosaccharide solution is used as a source of CO₂ for sugars oxygenation into CO₂, supply of air to the fitting is facilitated from oxygenator block 7.1.3, also connected with the supply rack for saturation of the water solution by oxygen and initiation of the sugars oxygenation to CO₂. Under these conditions the initiated photosynthesis allows gaining additional volume of oxygen, part of which will dissolve in water and finish oxygenation of sugars to CO₂, intensifying the photosynthesis. This combination of photosynthesis process and the CO₂ source is a biologic regulator of the photosynthesis process. In the course of sugars full oxygenation into CO₂ and subsequent photosynthetic process, CO₂ is consumed, and photosynthesis terminates. With the cessation of photosynthesis the generation of oxygen necessary for oxygenation of monosaccharides also stops.

### Daily Phase of Growing of Mother Solution

In case the monosaccharide solution is selected as a source of CO₂, the control system based on the operator's choice can facilitate the supply of the necessary amount of solution on the regular basis—once in 24 hours prior to day light, or in an intensive mode—when the specified amount of dissolved CO₂ is maintained. In case the source of CO₂ is gaseous CO₂, the control system automatically maintains the preset volume of CO₂ dissolved in water. If the source of CO₂ is a sugar solution, upon its introduction into the reactor the volume of media in the reactor increases. As a protection against reactor overfill, part of the unconditioned product from the upper level of the bioreactor is pumped into the unit for temporary storage of unconditioned product 7.1.23, which is used in the process for filling in a fresh portion of original water during daily extractions of the finished product.

During daylight the culture cells grow in the volume and gain weight, so that at the end of the daylight they are ready for cytokinesis. The need of the presence of light and carbon dioxide decreases. The control system sets time for initiation of the culture's preparation for rest and cytokinesis. Duration of this period is one hour. In proportion to this period the intensity of light flux in the segments of the reactor decreases, and stabilization ends with the supply of CO₂. The mode for degassing of CO2 dissolved in water through product aeration in the reactor switches on. With engaging the pump for product circulation from the bottom part of the reactor, the product is pumped into the fittings of washing distributor heads. Due to throttling of the flow and slight dispersion of CO₂ and O₂, the gases are expelled from water and removed from the bioreactor of mother solution; they pass into the unit for CO₂ and O₂ degassing from the water suspension 7.1.13 and further into the unit of technologies of the adsorption short cycle (KCA) and vacuum short cycle (VKCA) for separation of N₂, CO₂ and O₂, including the station for filling oxygen bottles 7.1.14.

After that, to prepare the culture to sleep the control system starts to feed in air or oxygen from the oxygenator for setting the specified concentration of oxygen dissolved in water in order to facilitate breathing of the culture during the night. After reaching the required concentration of oxygen the control system switches into automatic mode for maintaining the specified concentration of the dissolved oxygen during the night time; it also sets lower frequency of rotation of the sectional facility, thus facilitating stirring of the culture in a sparing mode.

At the time defined for the culture introduction into the bioreactor, or with the culture awakening, the control system switches off the mode for stabilizing the level of the dissolved oxygen, and restores the following parameters during the culture's awakening:
Intensity of light flux,
Preset daily frequency of rotation of the sectional facility, and
Preset amount of the dissolved CO₂ followed by stabilization of the culture growth during daytime; or the control system initiates batching liquid CO₂.

It means that at the moment of culture awakening all components of the photosynthetic process are recovered, and the process is cycled.

During the growth the culture reaches the preset optical density of the product, which requires extraction of finished culture from the mother solution's bioreactor. In accordance with the preset amount of product to be extracted the control system engages pumps for the product extraction from the bioreactor sending it to the block for storage of the finished suspension of Parachlorella KIEG 1904 7.1.16, and replaces the extracted suspension by a portion of unconditioned product from the block for temporary storage of unconditioned product 7.1.23, and a portion of fresh water. After replacement of the finished product the control system starts to supply the nutrition from the unit for preparation and supply of nutrition N 1 7.1.4 and 7.1.10.

From the storage unit of finished Parachlorella KIEG 1904 7.1.16 the suspension is supplied to the operational culture photobioreactor 7.1.2 and for shipment to customers. All processes in the photobioreactor 7.1.2 are similar.

Product as a Feed for Livestock—Suspension Based on Parachlorella KIEG1904

The plankton strain Parachlorella KIEG 1904 serves to obtain additional weight gain, increase milk yields, and improve reproduction and preservation of livestock. In poultry industry the strain is used in the parent breeding flock and also for feeding broilers. It is also used for biological rehabilitation of watersheds, decreasing of carbonate hardness of water, and production of dry biomass.

Device for cultivation of chlorella—module 7.1—is designed for the product manufacture and represents a basic module, which can be used to create facilities of any capacity.

The plankton strain Parachlorella KIEG 1904 is used for preparation of the product; the strain is characterized by high degree of light energy utilization and chemical composition of cells and products of cellular metabolism, including proteins, irreplaceable amino acids, vitamins, microelements, biologically active substances, which do not possess other water or land plants. The efficiency coefficient of photosynthetic active radiation is 3.6. Original technology is being designed for cultivation of chlorella with regard to biology and morphological uniqueness of the strain.

The advantage of the plankton form of chlorella is its exceptional adaptability to the conditions of aquaculture. The strain is unique by its criticality to the concentration of carbon dioxide. Therefore the CO2 saturation is adjusted either by biological way, or by supplying of carbon dioxide in the form of pure CO₂, under strict supervision of the control system. Production of the chlorella suspension requires minimum of chemical agents and energetic means. Besides, pollution of the environment is prevented, and the obtained product is ecologically pure.

The production of the chlorella suspension is a wasteless technology because the entire industrial product is used as a livestock feed.

Use of chlorella suspension for feeding animals enables to obtain additional weight gains up to 40% with livestock preservation up to 99%. Such results are obtained due to the fact that chlorella is a unique biological natural product. Neither aquatic, nor land plant has the same useful features as chlorella.

### Biochemical Characteristic of Parachlorella KIEG 1904

Chlorella suspension within the recommended use rates cannot be used as a substantial source of proteins in livestock diet. However, a complex of amino acids, vitamins, microelements ad bio-stimulators contained in chlorella ensure a full assimilation of the feedstuff, increase of live weight growth gain and preservation of young livestock.

One liter of chlorella suspension contains 1 g of biomass with amount of cells from 5 to 6 mil./per ml. The chlorella's effect on animals is decreased with strengthening or impoverishment in the density of the suspension's cells.

Parachlorella KIEG 1904 has the following biochemical composition in % of dry biomass:

| | |
|---|---|
| Protein | 55% |
| Lipids | 12% |
| Carbohydrates | 25% |
| Ashes | 8% |

The contents of amino acids in chlorella (g/kg of dry matter) are as follows:

| | |
|---|---|
| Glutamic acid | 31.84 |
| Aspartic acid | 25.66 |
| Leucin | 21.68 |
| Alanin | 20.13 |
| Valin | 17.58 |
| Glycin | 17.02 |
| Threonin | 13.66 |
| Fenylalanin | 12.06 |
| Serin | 11.60 |
| Isoleucin | 11.30 |
| Prolin | 9.78 |
| Lysin | 8.78 |
| Tyrosin | 8.25 |
| Arginin | 8.17 |
| Cystin | 7.53 |
| Tryptofan | 5.11 |
| Methionin | 4.82 |
| Histidin | 1.51 |

Chlorella suspension contains all known today vitamins. It is known that vitamins B12 and D are not synthesized by plants, but they are present in chlorella in substantial quantity. There are 7 to 9 mg of vitamin B12 and 100 mg of vitamin D in 100 g of dry chlorella. Chlorella's biomass contains as much vitamin C as a lemon; vitamin K plays a very important physiological role in the organism of an animal.

Contents of certain vitamins in chlorella (mg/g of dry matter) are as follows:

| | |
|---|---|
| Carotene | 1341 |
| Tocopherol (E) | 180 |
| Nicotinic acid | 140 |
| Riboflavin (B2) | 7.0 |
| Pyridoxine (B6) | 5.3 |
| Thiamine | 4.2 |

Due to introduction of microelements Fe, Cu, Co, Mn, Zn, I, Se into the nutrient medium, these elements are used by the chlorella strain, and animal organisms absorb microelements and organic substances more efficiently. Especially conditioned nutrient medium can be incorporated into the composition of the strain's chlorophyll, thus achieving a 100% digestibility of these microelements in the animal organism.
1. Phosphorus. Two thirds of phosphorus in vegetable additives is not assimilated by animals. In certain cases the additives used in feedstuffs make phosphorus non-absorbable by binding it up. It occurs more often with animals with a monogastric stomach, a single chamber as in case of pigs. The amount of phosphorus absorbed by the strain is calculated to provide a sufficient dose to the animal. Therefore, it is possible to decrease the overall volume of phosphorus in the feedstuff, and also production costs of breeding.
2. Copper sulfate CuSO₄. The amount of this component varies for different animals and period of feeding. It has been discovered that the use of copper in the amounts from 125 to 250 mg per 1 kg of feedstuff in the feeding batches increases the feed assimilation and growth of the weaner pigs. With content of copper exceeding 125 mg per 1 kg of feedstuff the efficiency decreases to 75-80%. It has been also proven that adding copper sulfate into the feeding batch has better effect for prevention of diseases than antibiotics. In fact, during fattening it is necessary to decrease the concentration of copper sulfate, because its influence over the growth in this period is decreasing, which may cause excess copper accumulation in the animal's liver.
3 Zinc oxide. It has been found out that zinc oxide has greater influence on the growth of the animal than copper during the animal's fattening. Zinc oxide is added to the nutrient medium in amounts ensuring the intensification of animal growth Zinc, likewise copper, is absorbed by the strain into the chlorophyll formula and is more digestible for animals. However, when doing zinc calculations it is necessary to analyze water for the content of zinc binding elements prior to its assimilation by chlorella. It has also been discovered that the contents of zinc plays an important role for formation and quality of boar's sperm.
4. Iron dichloride. Iron deficiency especially with piglets causes anemia. The shortage of iron originates with the piglets from their birth, because the sow is unable to pass her own supply through placenta. The piglets cannot gain iron from milk either. Supply of this element is facilitated by shots of iron dextran preparation. Further need for the preparation is determined by the amount of hemoglobin in blood. With concentration less than 10 mg per liter the shots are repeated. The introduction of iron dichloride into the nutrient medium of the strain enables to solve this issue through consumption of Parachlorella KIEG 1904. Also, as in case of zinc, the water content is analyzed, and if it contains iron, the added amount of iron dichloride is either adjusted, or completely omitted.
5. Selenium. During intensive growth of the animal the symptoms of white muscular disease can be observed owing to selenium deficiency. After slaughter the meat is pale in color. It starts to become calcified. After solution of this issue, as in case of other elements, the sodium selenite is introduced into the nutrient medium, in maximum concentration of 0.3 mg per 1 kg of feedstuff. It is important that selenium and vitamin E contained in chlorella are balanced, because they are interrelated.
6. Vitamin complex. The need for different vitamins for normal animal growth is an important element of the production economic expediency. This way vitamin E affects the quality of the sow's milk during nursing of piglets. If a weaner pig has enough vitamin E, it bears the stress associated with weaning from the sow much easier. Sows receiving sufficient amount of vitamin E incur inflammation of mammary gland less frequently. Research shows that feeding pigs with Parachlorella KIEG 1904 with vitamin A increases blood plasma, which consequently affects the amount of born piglets. Vitamin B 12—choline—similarly to vitamin A affects the number of born live piglets, and also the total number of the raised pigs. Parachlorella KIEG 1904 suspension contains a high volume of vitamin B 12.

The raw material source for LEC is:
Municipal waste generated by the population of the given area,
Industrial—housing sewage (sewage network) from the territory.

On the territory of LEC the collected solid municipal waste (SMW) is discharged into underground receiving chambers, which in ON condition contact the aerial atmosphere under slight pressure. From the chambers SMW passes into the module for waste preparation.

Industrial—housing sewage passes from the sewage network into the module for active sludge recovery from industrial and housing sewage.

Housing sewage is delivered to tanks, where anaerobic preparation of active sludge takes place, producing active sludge—thick organic matter—and technical water, which can be utilized for technology purposes of LEC and the facility as a whole.

In the preparation module active sludge is mixed with SMW, and is then supplied to the module of pyrolysis and gasification.

Synthetic gas, produced in the pyrolysis and gasification module, is supplied to the synthetic gas preparation module to generate the following gases: methane (CH₄), carbon dioxide (CO₂) and hydrogen monosulphide (H₂S). Methane is deodorized and sent through a commercial meter to the existing gas line to supply the given facility. Carbon dioxide is fed to the module for manufacture of the product of biotechnological importance. Hydrogen monosulphide is supplied into the module for elemental sulfur production implemented by oxygenation of hydrogen monosulphide H₂₅ during synthetic gas generation.

Gas, composed of carbon oxide (CO) and hydrogen (H2), is supplied into the module for power and heat generation to supply LEC with power and heat. Excess power and heat is passed through commercial meters into existing heat and power grids in the LEC facility.

Utilizing carbon dioxide from the module for carbon dioxide recovery out of combustion gases, collected during generation of power and heat, and also out of synthetic gas preparation module a “dry biomass”, i.e. matter recovered from photobioreactors through decanting or drying during production of raw materials for cosmetic and pharmacological products) is produced on the module for manufacture of the product of biotechnological importance. Oxygen generated in the course of photosynthesis is released from the given module into the atmosphere of the LEC facility.

### Utilization of Chlorella Suspension

The suspension of Parachlorella KIEG 1904 is utilized as a feed to reach for weight gains, preserve population of young livestock, increase of productivity of animal and poultry breeding, improvement of the livestock reproduction, and also for biological rehabilitation of watersheds, decrease of carbonate hardness of water, and production of dry biomass.

Utilization of chlorella suspension based on the strain provides the following results:
Increase of live weight gain: Calves 25-40%;
   Pigs—30-40%;
   Broilers—18-20%;
Preservation of population of young livestock: Calves—99%;
   Pigs—99%;
   Broilers—98%;
Laying ability by 10-15% and egg weight by 10%;
Chicken hatch rate by 25%
Increasing reproduction capabilities of animals;
Milk yields increased by 15-20%;
Decreased amount of non-productive artificial inseminations and normalized sexual cycle of cows with long service-period and long farrowing period.

Good results are gained by utilization of chlorella suspension for feeding of silkworms and reproduction of animals in fur industry (increased preservation of young animals and rate of growth, and improved quality of fur).

2. Means of biological rehabilitation of watersheds and eradication of blue-green algae.
3. Water softening agent for circulation water tanks in atomic power plants. Due to introduction of the strain into water with hardness of 21 German degrees the hardness value is reduced by 6 degrees and pH value increases from 6.8 to 9.9 units.

### Production Technology and Suspension Production of Parachlorella KIEG 1904

Production of the chlorella suspension is based on photosynthesis of microalgae performed in module 7.1 using artificial lighting and solution of carbon dioxide or pure liquid or gaseous CO₂.

The production process is uninterrupted; part of the suspension is extracted daily to feed livestock. Recovery of the strain takes place in the nutrition solution prepared according to a special recipe.

Chlorella cultivation runs throughout the year. Chlorella productivity does not depend on the season.

### Dosage and Period of Application of the Chlorella Suspension

Dosage and period of application of the chlorella suspension is shown with regard to other activity, i.e. creation of the growth rate effect and preservation of young livestock throughout the raising period.

| | Suspension dose | Feeding period, |
|---|---|---|
| Animal type | per capita, ml | days |
| Beef cattle | 1200-1500 | Daily |
| Cows | | |
| Before coupling | 1000 | 10 |
| Gestation period | 1000 | 30 |
| Lactation period | 1000 | 50 |
| Calves | 300-500 | 30 |
| Pigs | | |
| Sows before coupling | 1000 | 10 |
| Pregnant sows | 1000 | 30 |
| Prior to lactation | 1000 | 50 |
| Finishers | 1000 | Daily |
| Weaners up to 30 kg | 340-600 | Daily |
| Piglets | 200-300 | 21 |
| Poultry | | |
| Adult poultry | 30 | Daily |
| Young poultry | 5-30 | Daily |

During utilization of the strain as a feed no changes in technology of feeding, feeding dosage and animal breeding are performed. In course of algae application the use of antibiotics is eliminated (except for planned vaccinations, prophylactic measures and dehelmintization).

### Module 7.2

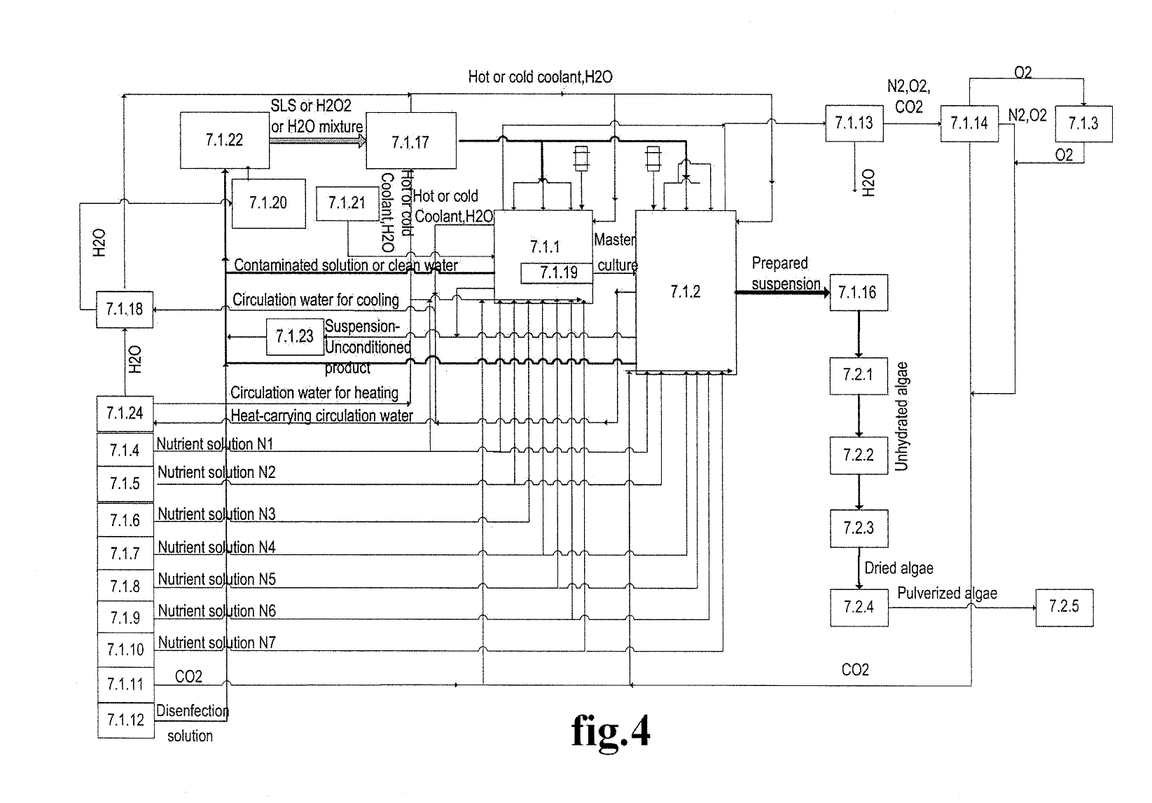

Processes in module 7.2 are analogous to processes in module 7.1. The difference lies in the way of the suspension processing into a dry biomass, see FIG. 4. The process of dry biomass production goes through several stages. At the beginning the algae suspension by concentration of 10-12 g/l is passed into the separation block 7.2.1, where water is separated from algal cells. Then the produced biomass passes to decanting block 7.2.2, where hard matter is removed, e.g. calcite impurities, if hard water is utilized. Elimination of calcium solves the problems which could emerge in regulation and measuring systems, or cause sterilization system clogging. Collected algae are then dried out in furnaces with low temperature in the block 7.2.3. The furnaces are drum-type microwave devices with uninterrupted operation that are designed for drying and final drying of algae. The devices have low specific power consumption, automatic turning of the product during drying, program control of the technology mode; they are reliable and easy to operate.

The principle of drying is based on common utilization of specially designed infrared lamps mounted on the entire drying surface, and convection process with pre-heated air. Product blasting system is designed in zones, i.e. each section represents separate climatic zone with regulated temperature, humidity and air flow velocity.

The algae then undergo micronization in the block 7.2.4, where they are processed into homogenous suspension in gas turbulent flow, i.e. algal particles disintegrate on their own due to changes in alternate air pressure and vibrations in cyclone. Low temperature is maintained to prevent decomposition of their active vitamin and protein contents. The algal cells are exploding in such atmosphere due to frictions. Cyclone collector and filter terminate the process producing fine dry powder (micronized algae). Compression and decompression of algal cells releases its protoplasmatic contents. (During traditional pulpification method of marine algae the cell walls are being just squeezed releasing mere pigments. Therefore, the precious ion content remains inside undamaged cell covering, and is unavailable for further processing). The algae are then, in the form of fine powder, supplied to the block 7.2.5, where the product is being packed.

### Module 7.3

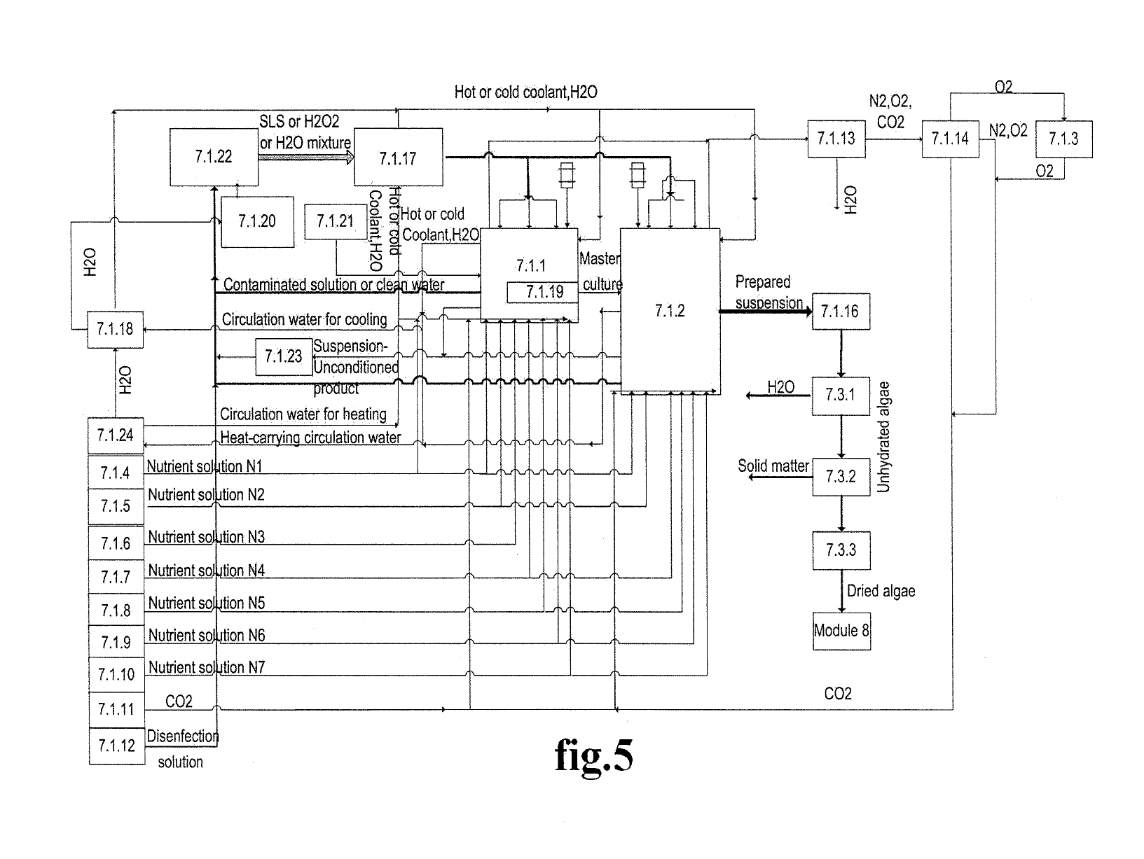

All processes in the module 7.3 are similar to processes in the module 7.2. The difference is in the absence of the micronization unit and presence of additional processes for finetuning of dry biomass for production of oil and coarse feed (see FIG. 5). At the beginning the algal suspension in concentration of 10-12 g/l passes into the separation block 7.3.1, where water is separated from algal cells. After that the produced biomass passes to decanting—block 7.3.2, where hard matter is removed, e.g. calcite impurities, in case of hard water is been used. Elimination of calcium solves the problems which could emerge in regulation and measuring systems, or cause sterilization system clogging. Collected algae are then dried in furnaces with low temperature in the block 7.2.3.

The drying in the module 7.3.3, as opposed to module 7.2, is different because the output product should have 30% humidity. It relates to the technology of oil extraction out of algae with no solvents used.

### Modification of Modules 7.2.2 and 7.2.3

The difference in technology for cultivation of Parachlorella KIEG 1904 for production of “dry” biomass is that instead of fresh water the operational bioreactor utilizes sewage water from industrial facilities:
Breweries,
Meat processing facilities,
Large poultry ranches,
Piggeries,
Complexes for cattle breeding

The given technology does not require the supply of nutrients N1 to N6 into the growing culture. All necessary nutrients are available in sewage received from the facilities. The process of recovery of dry biomass or vegetable oil is analogous to the foresaid production process.

### Module 8

The technology belongs to extraction of natural products, contained in biological materials and plants especially. The method enables to conduct extraction without the use of solvents, and ensures production of pure extract, free of any remains of solvents. Biological material—dry algae—is placed into a chamber free of solvents. The pressure is reduced intermittently. The biological material is concurrently exposed to microwave electromagnetic field. Mixture of vapors of extractant and extracted product is produced. The chamber is heated. Chamber heating, influence of the microwave field and pressure relief within the chamber are combined to enable product hydrodistillation of the mentioned biological material with water vapor. The chamber is heated to 100° C. The frequency of the electromagnetic field is app. 300 MHz. The power output is between 100 and 10 000 W/kg of the processed material.

### Module 9

The module is designed for production of aliphatic acids ester (methylester) out of algal oil. The raw material, algal oil, is pumped from module 8. The material is heated up to 35° C. in a heating economizer, using the heated pure biodiesel fuel. Then the material is heated using a burner through a heat exchanger to 60° C. Methoxide, recovered in the reactor for methoxide production, is fed into the re-etherification reactor. Re-etherification takes place in two reactors with cyclic operation. Raw glycerin is separated from bio-diesel fuel in a centrifuge, and is supplied into module 2. Mixture of methanol and bio-diesel fuel is heated to 70° C., and is then supplied through a feeder into the reactor for dry purge. Methanol is evaporated and condensed for repeated use. All other devices, which methanol vapors are produced in, create a closed system; methanol vapors are not released, but condensed, and are passed for repeated use. Contaminated bio-diesel fuel is refined by magnesium disilicate (1.5 kg per 10 000 liters) in cyclic system for dry purge. Magnesium plus collected particles and water are separated from bio-diesel fuel in special filtration system of dry purge. Hot refined bio-diesel fuel is cooled in a heater-economizer to 35° C., and then to 20° C. in a heat emitter. Pure bio-diesel fuel is supplied into balance tank for assessment of its quality, prior to supply of fuel into basic storage tank.

Operation of rotor-stator unit of the facility (reactor) is based on physical processes between rotor and stator. The raw material for processing is passed through the rotor-stator system and gains speed in centrifuge. The rotor system operates at velocity of 50 m/s in relation to stator. The raw material undergoes compression stage in chambers (between rotor and stator) with pressure of 10 bars. The compression time is 0.001 s. The raw material expands in the form of burst wave/compression jump, and runs into the next internal centrifuge. Parts of rotor and stator interfere up to 500 million times per second. In the process of microcavitation the product undergoes technological processing during energy transfer: electromotor—wall—rotor/stator—final product. This operation repeats one million times per second. This is the main difference between this device and other devices of similar design. This allows conducting the process of etherification under constant water pressure in the reaction zone with the object of preventing reaction from the left shift towards the initial products.

### Module 10

Replaceable filters are installed into the inert gas environment and, after pulverization, into the induction furnace 10. After smelting the mixture of gases, heated to melting temperature of the refractory metal contained in the initial raw material, is filtered through the system of zirconium filters, and poured into special moulds for cooling.

### Module 12

The module for synthesis of gasoline from synthetic gas comprises of the unit for synthetic gas preparation, reactor block, unit for stabilization and separation of the reaction products, and unit for preparation of regeneration gases. The unit for synthetic gas preparation is designed for mixing of the initial synthetic gas with recycled gas, compression of the recovered mixture to operating pressure, and supply of operational gas into the reactor block, separation of liquid reaction products from gaseous by high pressure separation. The reactor block is designed for synthesis of carbohydrates out of synthetic gas. The reactor block comprises of two identical reactor threads operating in parallel—thread A and thread B.

Each reactor thread represents 4 two-cascade reactors operating in “swing” scheme—3 reactors are producing gasoline, and the fourth reactor ensures the catalyst regeneration and backup. Among the three reactors, running in sequence in the gasoline synthesis stage, the products are partially cooled in recuperative heat exchangers.

From the reactor threads A and B the products are supplied to the unit for synthetic gas preparation with the object of liquid products high pressure separation from unreacted synthetic gas.

The unit for stabilization (separation) of the reaction products is designed for separation of condensed reaction products with separation of gaseous fractions, gasoline and water. Three-stage separator separates condensed products under low pressure, separating tank gases, and unstable carbohydrate catalyst and water condensate in the stabilization unit. Unstable catalyst is further subjected to rectification with separation of residual gases (stabilization gases), gasoline and heavy carbohydrate fraction with boiling point exceeding 200° C.

The unit for preparation of regeneration gases is designed for regeneration gases preparation and catalyst regeneration itself. To decrease the consumption of nitrogen supplied for catalyst regeneration the recycling of exhaust regeneration gases are prescribed in the technology process.

### LEGEND

1 Module of waste preparation
2 Module for waste pyrolysis and gasification (consisting of units 2.1-2.9)
2.1 Reactor of pyrolysis and partial gasification
2.2 Gasification reactor
2.3 Waste holder/feeder
2.3.0 Auger device
2.3.1 Chamber for waste batch forming
2.3.2 Steam generator
2.3.3 Block for material supply organization
2.4 Device for malt filtration from metals reduced in medium containing hydrogen and carbon (nickel, cobalt, copper, iron, bismuth, tin, lead, zinc, aluminum) with removable zirconium filters
2.5 Cyclone for mechanical purification of synthetic gas from carried particles of pyrocarbon and melted salts
2.6 Connecting manifold with arresting valves
2.6.1-2.6.8 Manifold
2.7 Heater
2.8 Damping tank for storage of contaminated synthetic gas
2.9 Pumping chamber for transferring salt melt into the pyrolysis reactor
2.11 Holder for bulk materials and other elements for correlation of quantitative composition of gas from module 2.8
3 Device for preparation of synthetic gas for various purposes
3.1 Device for priority separation of hydrogen on specifically selected membrane
3.11 Drying block
3.2 Gas tank
3.3 Device for priority separation of hydrogen monosulphide and carbon dioxide from gas on specifically selected membrane
3.4 Gas tank for temporary storage
3.5 Device for priority separation of carbon oxide from residual gas on specifically selected membrane
3.6 Gas tank for storage
3.7 Gas tank for storage
3.8 Gas tank
3.9 Gas tank for storage
3.10 Device for gas stirring
4 Device for extraction of carbon dioxide from combustion gases produced during generation of power and heat and fuel production
5 Device for power and heat generation from carbohydrate gases and glycerin recovered during production of aliphatic acids' esters
6 Device for production of elemental sulfur through oxygenation of hydrogen monosulphide during synthetic gas production
7 Facility for production of biotechnological products (comprises of units 7.1-7.3)
7.1 Facility for production of feeding suspension based on Parachlorella KIEG 1904 algae
7.1.1 Photobioreactor for mother solution
7.1.2 Photobioreactor for operational culture
7.1.3 Suspension oxygenator
7.1.4 Unit for preparation and supply of nutrient N 1
7.1.5 Unit for preparation and supply of nutrient N 2
7.1.6 Unit for preparation and supply of nutrient N 3
7.1.7 Unit for preparation and supply of nutrient N 4
7.1.8 Unit for preparation and supply of nutrient N 5
7.1.9 Unit for preparation and supply of nutrient N 6
7.1.10 Unit for preparation and supply of nutrient N 7
7.1.11 Unit for preparation and supply of CO₂ N1
7.1.12 Unit for preparation and supply of disinfecting solution N 1
7.1.13 Unit for degassing of CO₂ and O₂ from water solution of the suspension
7.1.14 Unit of technologies of the adsorption short cycle (KCA) and vacuum short cycle (VKCA) for separation of N₂, CO₂ and O₂, including the station for filling oxygen bottles
7.1.16 Unit for storage of the finished suspension of Parachlorella KIEG 1904
7.1.17 Unit for heating of the suspension and original water
7.1.18 Unit for suspension cooling
7.1.19 Unit for supply of the mother solution into the photobioreactor's operational culture
7.1.20 Unit for original water treatment
7.1.21 Unit for preparation and supply of basic strain to the mother solution's photobioreactor
7.1.22 Unit for preparation and supply of disinfecting solution
7.1.23 Unit for temporary storage of unconditioned product
7.1.24 Unit for reception of water from source
7.2 Facility for production of the suspension for regeneration of watersheds and eradication of blue-green algae
7.2.1 Unit for water separation from algae cells
7.2.2 Block for decanting with the object of removing solid matter
7.2.3 Block of low-temperature furnaces
7.2.4 Micronization block
7.2.5 Coating block
7.3 Facility for dry biomass production
7.3.1 Unit for water separation from algal cells
7.3.2 Block for decanting with the object of removing solid
7.3.3 Drying unit
8 Facility for production of oil out of dry algae biomass
9 Device for production of aliphatic acids' esters (FAME) out of algae oil
9.1 Unit for glycerol extraction
10 Module for smelting of metals recovered during chemical reduction by hydrogen in module for pyrolysis and gasification in induction furnaces
11 Facility for production of active sludge out of manufacturing water and sewage
12 Module for gasoline synthesis out of synthetic gas

1. Method of organic waste processing characterized by the fact that module (1) receives all organic waste, while separation to glass and metals takes place in module (1) and remains of food and other waste are minced and stabilized by supply of air and aerobian bacteria, the waste passes out through the biotunnel, where temperature is raised and water evaporates, which is being condensed and re-used for other purposes, while the waste, passing through biotunnel, is compressed into pellets, which are transported using carbon dioxide into the loading device, from which it is fed by auger into dispenser (2.3), where the waste is compressed again, then the compressed waste is expelled by carbon dioxide into the reactor (2.1), when this reactor contains melt of various salts at temperatures from 900 to 1000° C., while in the reactor (2.1) pyrolysis to individual elements C, H, N, S, O₂ takes place and methane and other gases are generated, while the composition of gases depends on the pressure inside the pyrolysis chamber, when the amount of methane is modified using externally regulated pressure, when metal oxides, being part of the wastes from thermal cracking, are reducer to pure metals through melt, while the top of the reactor (2.1) contains gasses and the bottom part—carbon and pure metals, while pressure also adjusts the level of melt and also ensures mechanical stirring, while after finished thermal cracking, duration of which is determined through calculation, a valve opens, connecting manifold (2.6), and melt runs under pressure into the reactor (2.2), while reactor (2.1) is connected by two manifolds with gasification reactor (2.2), while gasses from the top part of the reactor (2.1) pass through manifold (2.6.1) into the bottom part of the reactor (2.2) and melt at the reactor (2.1) passes through manifold (2.6) into the bottom part of the reactor (2.2), while steam is being added into the top manifold (2.6.1) from the steam generator (2.10), and in the reactor (2.2) gasification takes place in the presence of steam at temperatures between 940 to 1000° C., generating H, CO, CO₂, ¾S, NH₄, CH₄ and other carbohydrate compounds, according to the presence of elements, while the pressure regulates the contents of methane and other carbohydrates up to butane, where gases pass to the upper part of the reactor chamber (2.2) and through manifold (2.6.2) pass into the cyclone (2.5), where they are mechanically refined, while a part returns into the holder (2.3), where pure gas flows through manifold (2.6.3) into the holder (2.8), where analysis for the presence of dangerous FH gases or dioxin is performed, and in case of dangerous gases detection the valve on manifold (2.6.5) closes and valve on manifold (2.6.4) opens and the contaminated gas returns into the reactor (2.1), supply of waste into the holder (2.3) is terminated and from the holder of elements for gas quality correction (2.11) hydroxides are being added into the holder (2.3) for neutralization, and the entire process is repeated, and after gasification in reactor (2.2) is completed, valve on manifold (2.6.6) opens and increased level of melt automatically partially transfers to the device (2.4) for filtration, where zirconium filter with apertures of different diameters, through which the melt passes and the apertures capture the metals, and saturated filters are placed into induction furnace (10), where the filter is pulverized in inert environment to powder, which serves as raw material for further processing, while the melt passes from device (2.4) through the manifold (2.6.7) into the pump chamber (2.9), from which the melt is expelled through manifold (2.11) into the reactor (2.1), where the cycle is completed, whereas sewage water is considered a source of raw material, which passes into the holder (11), where oxygenation by anaerobic bacteria takes place, producing sludge and technical water, and sludge can be blended in the holder for liquid waste (2.3) either together with solid waste, or separately.

2. A facility for complex processing of organic waste, comprising of waste preparation module, which includes waste separation, waste mixing and biotunnel, another module for waste pyrolysis and gasification, including reactor for pyrolysis and partial gasification connected into the gasification reactor, while above the reactor for pyrolysis and partial gasification a liquid waste tank is mounted, serving as a feeder, characterized by the fact, that reactor (2.1) for pyrolysis and partial gasification and reactor (2.2) for gasification contain melt of salts and are connected into the device (2.4) for melt filtration with heater (2.7), where the device (2.4) has on connected module (10) for metal smelting on one side and pumping chamber (2.9) on the other, for transferring salt melt into the reactor (2.1), while the reactor (2.2) is connected by manifold (2.6) with cyclone (2.5) for mechanical purification, and through manifold (2.6) the pumping chamber (2.9) is also connected with the reactor (2.1), and further by this manifold the reactor (2.1) is connected with the reactor (2.2), where the cyclone (2.5) is connected with the damping tank (2.8) for storage of contaminated synthetic gas, which is connected with the reactor (2.1) through a manifold for extraction of raw gas and module (3) for preparation of synthetic gas for various purposes, while from module (3) manifold passes to module (4) and from module (3) manifold also leads into module (5) for power and heat generation, and through manifold to module (6) for elemental sulfur production, while module (4) is connected with branching manifold with the module for production of biotechnological products from device (7.1) for production of feeding suspension based on algae Parachlorella KIEG 1904, device (7.2), for production of algal suspension for regeneration of watersheds and eradication of blue-green algae, and device (7.3) for production of dry biomass, while device (7.3) is connected with device (8) for production of oil out of dry algae biomass, which is further connected with device (9) for production of aliphatic acids' esters out of algal oil, while device (9) is branched into block (9.1) for glycerol extraction, from which the manifold connects to module (5) designed for generation of power and heat.

3. Bio-technological products produced on the facility under claim 1, containing the algal strain Parachlorella KIEG 1904, will be used for feeding livestock.

4. Biological suspensions produced by a method under claim 1 and on the facility under claim 2 will be used for biological rehabilitation of water reservoirs and eradication of blue-green algae.

5. Biological suspensions, produced by a method subject to claim 1 and on device subject to claim 2, will be used for water treatment.

6. The equipment under claim 2 will serve for production of biological suspension based on the strain Parachlorella KIEG 1904.

7. The equipment under claim 2 will be used for complex utilization of the technologies for waste processing and bio-technologies through building of underground compounds in residential areas (minimum 5000 residents, maximum 20 000 residents), i.e. Local Energetic Compounds (LEC).
